(19) United States
(12) Patent Application Publication
(10) Pub. No.: US 2019/0314255 A1
(43) Pub. Date: Oct. 17, 2019

(54) Cleansing Concentrate Composition

(71) Applicant: Lonza Inc., Morristown, NJ (US)

(72) Inventor: Elias Pambou, Blackley (GB)

(21) Appl. No.: 16/371,662

(22) Filed: Apr. 1, 2019

Related U.S. Application Data
(60) Provisional application No. 62/658,202, filed on Apr. 16, 2018.

(51) Int. Cl. A61K 8/37; A61Q 19/10; A61Q 19/00; A61K 8/02; A61K 8/04; A61K 8/06; A61K 8/34; A61K 8/49; A61K 8/92

(52) U.S. Cl. CPC A61K 8/375; A61Q 19/10; A61Q 19/007; A61K 8/0208; A61K 8/922; A61K 8/062; A61K 8/34; A61K 8/498; A61K 8/042

(57) ABSTRACT

The invention provides a cleansing concentrate composition for use as a lotion, solution, cream, gel, solid or wet wipe saturant having (a) a first emulsifier selected from group consisting of a polyglyceryl fatty ester, an ethoxylated fatty alcohol, an ethoxylated fatty acid, an ethoxylated sorbitan ester and a mixture thereof, having an HLB between 1 and 9; (b) a second emulsifier selected from group consisting of a polyglyceryl fatty ester, an ethoxylated fatty alcohol, an ethoxylated fatty acid, an ethoxylated sorbitan ester and a mixture thereof having an HLB between 9-16; (c) an emollient; (d) a preservative system and (e) an aqueous solvent. The composition may be free of any humectants and may be formulated for end-user personal care, hygiene, consumer care and cosmetic products such as wet wipes, hygiene wipes, baby wipes, make-up removal solutions, moisturizers, facial cleansers and scalp/hair care products.

The present application is based upon and claims priority to U.S. Provisional Application Ser. No. 62/658,202, filed on Apr. 16, 2018, which is incorporated herein by reference

### FIELD OF INVENTION

The disclosure relates to a cleansing concentrate composition for use as a formulation base, lotion, solution, cream, gel, solid or wet wipe saturant.

### BACKGROUND OF THE INVENTION

Current concentrate solutions on the market are often formulated by using PIT (Phase-inversion temperature) or PIC (Phase-inversion concentration) emulsification methods to form stable water-in-oil (W/O) concentrate solution, or microemulsions. However, the problem of formulating products using such systems is that they generally require an input of energy to convert the solution from W/O to an oil-in-water (O/W) system upon dilution. Additionally, such systems require formulations to be diluted through a method of catastrophic phase inversion or undergo a temperature change to obtain a stable O/W system in its final form. The phase-inversion process results in the size modification of the oil-droplets and dictates the morphology of the final emulsion. It is very cost ineffective and energy consuming to use a system which outputs a large amount of heat and mechanical energy to carry out the phase-inversion and control both the oil-droplet size and stability of a formulation (i.e. by use of high pressure homogenization). Furthermore PIT and PIC emulsification processes require a specific set of instructions or formulation steps to be followed in order to generate a stable product

Microemulsions are thermodynamically stable, single-phase systems that can be diluted in aqueous media under mild agitation. The formation of microemulsions, however, depends strongly on the balance between a surfactant's hydrophilic and hydrophobic properties, determined by a chemicals structure, temperature, salinity and the correct combination of co-solvents or co-surfactants. A further disadvantage is a limited stability range and use range, by severely restricting the final end-user formulation of the concentrate, such as the addition of preservatives or fragrances.

There is a need in the art to provide a cleansing concentrate composition, for use as a concentrate base lotion, solution, cream, gel, solid or wet wipe saturant, which is highly stable at highly concentrated O/W compositions for end-user formulations, without the need for formulation systems or formulation restrictions such as those described above. Additionally, there is a need in the art to provide an O/W system so that no phase change, inversion, change in form or instability is undertaken assuring the dilution process.

The present invention meets this need in the art by providing a simpler, highly stable and versatile composition via cold-processible means, resulting in lower manufacturing cost, less energy and time consumption, and also provides all of the benefits of formulations containing additional ingredients such as humectants, viscosity modifiers and emollients, including, for example, rheological, aesthetic spreading and drying properties, without affecting the overall properties of the concentrate.

### SUMMARY OF THE INVENTION

The present invention provides a concentrate composition for use in personal care, cosmetics, consumer care, and hygiene applications.

In one embodiment, the present invention provides a cleansing concentrate composition for use as a concentrate base, lotion, solution, cream, gel, solid or wet wipe saturant comprising;
(a) a first emulsifier selected from group consisting of a polyglyceryl fatty ester, an ethoxylated fatty alcohol, an ethoxylated fatty acid, an ethoxylated sorbitan ester and a mixture thereof, having an HLB between 1 and 9;
(b) a second emulsifier selected from group consisting of a polyglyceryl fatty ester, an ethoxylated fatty alcohol, an ethoxylated fatty acid, an ethoxylated sorbitan ester and a mixture thereof having an HLB between 9-16;
(c) an emollient;
(d) a preservative system; and
(e) an aqueous solvent.

In one embodiment, the wherein the first and second emulsifier are each present in an amount between 2 w/w %-30 w/w %, based on the weight of the composition; the emollient is present in an amount between 15 w/w %-80 w/w %; based on the weight of the composition; the preservative system present in an amount from 0.01 w/w %-10 w/w %, based on the weight of the composition; and the aqueous solvent present in an amount from 12 w/w %-55 w/w %, based on the weight of the composition.

In one embodiment, the first or second emulsifier comprises more than one of a polyglyceryl fatty ester, an ethoxylated fatty alcohol, an ethoxylated fatty acid and mixtures thereof.

In another embodiment, the ratio of the first emulsifier to the second emulsifier is about 1:10 to 10:1.

In one particular embodiment, the first and second emulsifier each comprises one or more compound of a polyglyceryl fatty ester.

In one particular embodiment, the polyglyceryl fatty ester is derived from (1) a polyglycerol component built up from 2 to 12 molecules of glycerol, based on an average, and (2) a fatty acid comprising a caprylic acid, capric acid, lauric acid, myristic acid, palmitic acid, stearic acid, arachidic acid, behenic acid, lignoceric acid, cerotic acid, oleic acid, decaoleic acid or mixtures thereof.

In another embodiment, (a) the first emulsifier comprises a polyglyceryl fatty ester wherein the polyglyceryl fatty esters comprises one or more of a polyglyceryl-decaoleate, polyglyceryl-3 monostearate, polyglyceryl-6 distearate, or polyglyceryl-3 tetraoleate, and the (b) second emulsifier comprises a polyglyceryl fatty ester, wherein the polyglyceryl fatty esters comprises polyglyceryl-10 stearate, polyglyceryl-10 oleate, polyglyceryl-10 dipalmitate, polyglyceryl-10 caprylate/caprate, or polyglyceryl-10 laurate.

In one embodiment, the emollient comprises a wax or an oil, and wherein the oil comprises an ester oil, an ether oil, or a mixture thereof.

In one particular embodiment, the ester oil comprises a dialkyl tartrate, dialkyl carbonate, marula oil, sunflower seed oil, safflower seed oil, coconut oil, capryl/caprylic triglyceride, linseed oil, canola oil, cottonseed oil, soybean oil, or mixture thereof.

In one embodiment, the preservative system comprises one or more of a benzoic acid or salt thereof, benzyl alcohol, sorbic acid or salt thereof, parabens, dehydroacetic acid, sodium dehydroacetate, bronopol, triclosan, imidazolidinyl urea, PHMB (polyhexamethylene biguanide), phenoxyethanol, DMDMH (1,3-Dimethylol-5,5-dimethylhydantoin), isothiazolones, chlorhexidine, diazolidinyl urea, chlorphenesin, sodium hydroxymethylglycinate, benzethonoium chloride, ethylhexylglycerol, IPBC (iodopropynyl butylcarbamate), salicylic acid or salt thereof, sodium benzoate, calcium gluconate, D-glucono1,5-lactone, glycerin, or natural acid.

In one embodiment, the aqueous solvent comprises water, aqueous alcohols, ammonia water, acid solutions, salt solutions, water-miscible organic solvents, or mixtures thereof.

In one particular embodiment, the aqueous solvent comprises water.

In one embodiment, the present invention further comprises a humectant, wherein the humectant is present in an amount from 10 w/w %-45 w/w %, based on the total weight of the composition.

In another embodiment, the humectant is present in an amount less than 10 w/w %.

In a further embodiment, the cleansing concentrate composition is essentially free of a humectant.

In one particular embodiment, the present invention provides a cleansing concentrate composition wherein:
a) the first emulsifier comprises a polyglyceryl fatty ester and wherein the polyglyceryl fatty esters comprises one or more of a polyglyceryl-10 decaoleate, polyglyceryl-3 monostearate, polyglyceryl-6 distearate, polyglyceryl-3 tetraoleate, and the (b) second emulsifier comprises a polyglyceryl fatty ester, wherein the polyglyceryl fatty esters comprises polyglyceryl-10 stearate, polyglyceryl-10 oleate, polyglyceryl-10 dipalmitate, polyglyceryl-10 caprylate/caprate, or polyglyceryl-10 laurate;
(c) the emollient comprises an oil, wherein the oil comprises an ester oil, an ether oil or mixtures thereof;
(d) the preservative system comprising one or more of a dehydroacetic acid, benzyl alcohol, sorbic acid, salicylic acid, benzoic acid, sodium benzoate, calcium gluconate, D-glucono1,5-lactone, glycerin, phenoxyethanol, DMDMH (1,3-Dimethylol-5,5-dimethylhydantoin), sodium dehydroacetate, or iodopropynyl butylcarbamate (IPBC);
(e) the aqueous solvent comprises water; and
   wherein the concentrate composition totals up to 100 w/w %, based on the weight of the composition.

In another embodiment, the cleansing concentrate composition further comprises a co-surfactant, fragrance, viscosity modifier, moisturizing agent, antioxidant, active pharmaceutical ingredient, booster, potentiator, biocidal agent, cosmetic active, sun-protectant, UV-protectant, biological active, occlusive agent or mixtures thereof.

In another aspect, the present invention provides a wet wipe impregnating formulation for personal care, cosmetics or hygiene products comprising (a) a concentrated composition according to claim 1, and (b) an aqueous solvent comprising water, an aqueous alcohol or a mixtures thereof.

In one embodiment, the wet wipe impregnating formulation comprises 0.1 w/w %-50 w/w %, by weight of the concentrate composition, based on the weight of the formulation.

In one embodiment, the wipe substrate comprises a woven, a nonwoven material or mixtures thereof.

In one particular embodiment, the wet wipe impregnating formulation and the wipe substrate in a weight ratio of about 10:1 to 1:1.

### DETAILED DESCRIPTION

It has now been surprisingly found that a highly stable, highly concentrated O/W a cleansing concentrate for use as a concentrate base, lotion, solution, cream, gel, solid or wet wipe saturant may be formed by combining one or more emulsifiers having an HLB of 1-16, an emollient, a preservative system, and aqueous solvent.

The invention provides a concentrate composition with good aesthetic properties for personal care applications. The end-user formulation may use the cleansing concentrate or a diluted form of the concentrate in an aqueous solvent (such as water, alcohols), add preservatives, fragrance and other additives, if desired, to form a fully finished and stable o/w system, via cold-processing, for personal care applications.

Additionally, the aesthetic properties and rheological behavior of polyglycerol esters means that additional components (such as humectants, viscosity modifiers), although not required; may be added to the end user formulation, if desired, without affecting the properties of the formulation itself. The highly stable O/W concentrate composition may be formed where the larger emollient portion of the formulation (by weight) makes up the dispersed droplet phase with the smaller water portion making up the continuous phase. Hence, an oil-dominated 0/1A/concentrate composition is formed that may be easily diluted by the addition of an aqueous solvent.

Polyglyceryl esters are used as naturally derived, multifunctional emulsifiers to create high internal phase technology emulsions in the form of highly concentrated oil-in-water (o/w) emulsion system. The o/w systems are defined as systems where water, or a water-soluble, or aqueous solution, makes up the continuous phase, and oil (or emollient) as the dispersed phase; in that oil droplets created by an emulsification process (the dispersed phase) can be stabilized (or emulsified) by an emulsifier in such a way that they are dispersed in a water-based solution (the continuous phase). The presence of an emulsifier is required to prevent the interaction and coalescence of the oil droplets which could eventually lead to separation of the oil and water phases, and therefore an unstable product. Generally, for o/w emulsions the water content (continuous phase) is much greater than 50 w/w % however in this embodiment we show that the combination of 2-or-more polyglyceryl esters allows for the formulation of high internal (or dispersed) phase o/w emulsions where the concentration of water is <50 w/w %.

The present invention provides a concentrate composition for use in personal care, cosmetics, consumer care, and hygiene applications. Such compositions are usually diluted, dissolved or dispersed in end-user formulation products such as wet wipes, hygiene wipes or hygiene lotions or solutions, baby wipes, makeup removal solutions, cleansing or moisturizing lotions, creams, and/or facial cleansers. They may also be used as concentrates in shampoos, conditioners, and/or scalp/hair care products.

One unique aspect of the invention is that the concentrate composition, for use as a concentrate base, lotion, solution, cream, gel, solid or wet wipe saturant, is essentially free of any humectant; and the composition remains very stable over an extended period of time.

In accordance to the present invention, the term “essentially free” herein, refers to a composition containing less than 0.5 w/w % of a particular substance. Suitably, a composition containing less than 0.2 w/w % of a particular substance.

The term “HLB” herein, refers to “hydrophilic-lipophilic balance”. The HLB value expresses the water and oil solubility of, in particular, non-ionic, emulsifiers (i.e. it expresses the lipophilic and hydrophilic properties of an emulsifier, which are determined by the different parts of the emulsifier molecules). The higher the HLB of an emulsifier, the more hydrophilic and water soluble it is. According to the Griffiths method, HLB is calculated via: HLB =(molecular weight of hydrophilic head-group/total molecular weight)*20.

The present invention encompasses a cleansing concentrate for use as a concentrate base, lotion, solution, cream, gel, solid or wet wipe saturant containing
(a) a first emulsifier selected from group consisting of a polyglyceryl fatty ester, an ethoxylated fatty alcohol, an ethoxylated fatty acid, an ethoxylated sorbitan ester and a mixture thereof, having an HLB between 1 and 9;
(b) a second emulsifier selected from group consisting of a polyglyceryl fatty ester, an ethoxylated fatty alcohol, an ethoxylated fatty acid, an ethoxylated sorbitan ester and a mixture thereof having an HLB between 9-16;
(c) an emollient;
(d) a preservative system; and
(e) an aqueous solvent.

It may be desired to have a cleansing concentrate for use as a concentrate base lotion, solution, cream, gel, solid or wet wipe saturant. Typically, the first or second emulsifier are each present in an amount between 1 w/w %-30 w/w %, based on the weight of the composition. Suitably, the first or second emulsifier are each present in an amount between 2 w/w %-30 w/w %, based on the weight of the composition. Desirably, the first or second emulsifier are each present in an amount between 3 w/w %-20 wlw %, based on the weight of the composition. The cleansing concentrate typically contains (a) the first emulsifier and (b) the second emulsifier present in an amount between 2 w/w %-30 w/w %, based on the weight of the composition; (c) an emollient present in an amount between 15 w/w %-80 w/w %; based on the weight of the composition; (d) a preservative system present in an amount from 0.01 w/w %-10 w/w %, based on the weight of the composition; and (e) an aqueous solvent present in an amount from 12 w/w %-55 w/w %, based on the weight of the composition.

Additionally, another suitable cleansing concentrate for use as a concentrate base, lotion, solution, cream or wet wipe saturant whereby the first and second emulsifier are each present in an amount between 3 w/w %-20 w/w %, based on the weight of the composition; the emollient is present in an amount between 40 w/w %-80 w/w %; based on the weight of the composition; and the preservative system present in an amount from 0.5 w/w %-5 w/w %, based on the weight of the composition.

Suitable emulsifiers for the first or second emulsifier may include more than one compound selected from group consisting of a polyglyceryl fatty ester, an ethoxylated fatty alcohol, an ethoxylated fatty acid, an ethoxylated sorbitan ester, mixtures thereof, and the like.

Typically, the ratio of the first and second emulsifier may be between about 1:10 to 10:1. For example, the weight ratio of more than one ethoxylated fatty alcohol to more than one polyglyceryl esters; or the weight ratio of one polyglyceryl ester to another polyglyceryl ester, in a cleansing concentrate composition, may be between about 1:10 to about 10:1. In another example, the weight ratio of first emulsifier(s) to the second emulsifier(s) is in a ratio of about 1:10 to about 10:1. Suitably, the weight ratio of the first and second emulsifier may be from about 1:5 to about 10:1, or from about 1:1 to about 5:1.

Typically, the fatty alcohol present in the concentrate composition comprises, for instance, any suitable fatty alcohol having a carbon chain length from about C₄-C₃₆ carbon atoms, such as from about C₆-C₂₈ carbon atoms.

Suitably, the fatty alcohol include, but are not limited to, a mixture of branched, unbranched, saturated or unsaturated fatty alcohols. Examples of fatty alcohols include, but are not limited to, capryl alcohol, pelargonic alcohol, capric alcohol, lauryl alcohol, myristyl alcohol, cetyl alcohol, palmitoleyl alcohol, stearyl alcohol, oleyl alcohol, arachidyl alcohol, behenyl alcohol, erucyl alcohol, lignoceryl alcohol, ceryl alcohol, or mixtures thereof, and the like.

Examples of ethoxylated sorbitan esters include, but are not limited to, polyoxyethylene (20) sorbitan monolaurates, polysorbate (40) monopalmitates, polysorbate (60) monostearates, polysorbate (80) monooleates, polysorbate (81) sorbitan monooleate, polysorbate (85) sorbitan trioleates, or mixtures thereof, and the like.

Typical polyglyceryl esters useable in the present invention may be formed from saturated, unsaturated, natural or synthetic fatty acids, mixtures thereof, and the like. For instance, examples of saturated fatty acids include, but are not limited to, caprylic acid, capric acid, lauric acid, myristic acid, palmitic acid, stearic acid, arachidic acid, behenic acid, lignoceric acid, cerotic acid, oleic acid, decaoleic acid, combinations thereof, derivatives thereof, and the like.

Furthermore, the polyglyceryl esters are derived from (a) a polyglycerol component built up from 2 to 12 molecules of glycerol, based on an average, and (b) a fatty acid comprising of a caprylic acid, capric acid, lauric acid, myristic acid, palmitic acid, stearic acid, arachidic acid, behenic acid, lignoceric acid, cerotic acid, oleic acid, decaoleic acid, or mixtures thereof, and the like.

Typical examples of polyglyceryl ester, having an HLB value between 1-9 or 9-16, includes, but are not limited to, polyglyceryl monodecaoleate such as polyglyceryl-10 decaoleate; polyglyceryl monooleate such as polyglyceryl-2-monooleate, polyglyceryl-3 monooleate, polyglyceryl-4 monooleate, polyglyceryl-3 tetraoleate, polyglyceryl-6 monooleate, or polyglyceryl-10 monooleate; polyglyceryl dioleate such as polyglyceryl-2 dioleate, polyglyceryl-3 dioleate, polyglyceryl-5 dioleate, polyglyceryl-6 dioleate or polyglyceryl-10 dioleate; polyglyceryl trioleate such as polyglyceryl-5 trioleate or polyglyceryl-10 trioleate; polyglyceryl tetraoleate such as polyglyceryl-2 tetraoleate, polyglyceryl-6 tetraoleate, or polyglyceryl-10 tetraoleate; polyglyceryl pentaoleate such as polyglyceryl-4 pentaoleate, polyglyceryl-6 pentaoleate, or polyglyceryl-10 pentaoleate; polyglyceryl heptaoleate such as polyglyceryl-6 heptaoleate, polyglyceryl-10 heptaoleate; polyglyceryl monostearate such as polyglyceryl-2 monostearate, polyglyceryl-3 monostearate, polyglyceryl-4 monostearate, polyglyceryl-5 monostearate, polyglyceryl-6 monostearate or polyglyceryl-10 monostearate; polyglyceryl distearate such as polyglyceryl-2 distearate, polyglyceryl-3 distearate, polyglyceryl-4 distearate, polyglyceryl-6 distearate, or polyglyceryl-10 distearate; polyglyceryl tristearate such as polyglyceryl-4 tristearate, polyglyceryl-5 tristearate, polyglyceryl-6 tristearate, or polyglyceryl-10 tristearate; polyglyceryl tetrastea rate such as polyglyceryl-2 tetrastearate; polyglyceryl pentastearate such as polyglyceryl-4 pentastearate, polyglyceryl-6 pentastearate, or polyglyceryl-10 pentastearate; polyglyceryl heptastearate such as polyglyceryl-10 heptastearate; polyglyceryl isostearate such as polyglyceryl-2 isostearate, polyglyceryl-3 isostearate, polyglyceryl-4 isostearate, polyglyceryl-6 isostearate, or polyglyceryl-10 isostearate; polyglyceryl diisostearate such as polyglyceryl-2 diisostearate. polyglyceryl-3 diisostearate, polyglyceryl-4 diisostearate, polyglyceryl-6 diisostearate, polyglyceryl-10 diisostearate, or polyglyceryl-15 diisostearate; polyglyceryl triisostearate such as polyglyceryl-2 triisostearate, polyglyceryl-3 triisostearate, polyglyceryl-5 triisostearate, polyglyceryl-10 triisostearate; polyglyceryl tetraisostearate such as polyglyceryl-2 tetraisostearate; polyglyceryl caprylate such as polyglyceryl-2 caprylate, polyglyceryl-3 caprylate, polyglyceryl-4 caprylate, polyglyceryl-6 caprylate, or polyglyceryl-10 caprylate; polyglyceryl dicaprylate such as polyglyceryl-5 dicaprylate; polyglyceryl sesquicaprylate such as polyglyceryl-2 sesquicapyrlate; polyglyceryl octacaprylate such as polyglyceryl-6 octacaprylate; polyglyceryl caprate such as polyglyceryl-2 caprate, polyglyceryl-3 caprate, polyglyceryl-4 caprate, polyglyceryl-5 caprate, polyglyceryl-6 caprate, polyglyceryl-10 caprate: polyglyceryl dicaprate such as polyglyceryl-3 dicaprate or polyglyceryl-6 dicaprate; polyglyceryl caprylate/caprate such as polyglyceryl-4 capyrlateicaprate, polyglyceryl-6 caprylate/caprate, or polyglyceryl-10 caprylate/caprate; polyglyceryl palmitate such as polyglyceryl-2 palmitate, polyglyceryl-3 palmitate, polyglyceryl-6 palmitate or polyglyceryl-10 palmitate; polyglyceryl dipalmitate such as polyglyceryl-6 dipalmitate or polyglyceryl-10 dipalmitate; polyglyceryl tetrabehenate such as polyglyceryl-6 tetrabehenate; polyglyceryl myristate such as polyglyceryl-6 myristate or polyglyceryl-10 myristate; polyglyceryl rincinoleate such polyglyceryl-6 polyricinoleate or polyglyceryl-10 ricinoleate; other derivatives thereof, or mixtures thereof, and the like.

Suitably, the cleansing concentrate contains a first and second emulsifier whereby the first and second emulsifier each comprises one or more compound of a polyglyceryl fatty ester.

Suitably, a variety of polyglyceryl esters in various forms may be used, specifically whereby the (a) first emulsifier includes, but not limited to one or more of a polyglyceryl-10 decaoleate, polyglyceryl-3 monostearate, polyglyceryl-6 distearate, or polyglyceryl-3 tetraoleate, and the (b) second emulsifier comprises a polyglyceryl fatty ester, wherein the polyglyceryl fatty esters comprises polyglyceryl-10 stearate, polyglyceryl-10 oleate, polyglyceryl-10 dipalmitate, polyglyceryl-10 caprylate/caprate, or polyglyceryl-10 laurate.

The composition for a cleansing concentrate for use as a concentrate base, lotion, solution, cream, gel, solid or wet wipe saturant, may contain an emollient, whereby the emollient is a wax, oil, mixtures thereof, and the like.

Typically, the emollient comprises a wax or an oil, whereby the wax or oil is present in an amount between 15 w/w %-80 w/w %; based on the weight of the composition. Suitably the emollient is present in an amount between 40 w/w %-80 w/w %; based on the weight of the composition.

The term “waxes” herein, refers to naturally-occurring or synthetic waxes, of which typical examples include stearyl alcohol, hydrocarbon waxes, waxes of plant or animal origin, their synthetic analogues or derivatives, or silicone waxes. Waxes are widely available, and by suitable selection of the waxes themselves and their concentrations in the formulation can effectively obtain either a soft solid or a firm solid. Conventionally, waxes are applied to a variety of materials and mixtures which have similar physical properties, namely that: they are solid at about 30° C. to about 40° C.; they melt to a mobile liquid at a temperature above 30° C., and below 95° C.; generally in a temperature range of 40° C. to about 90° C.; and they are water-insoluble and remain water-immiscible when heated above their melting point.

A variety of waxes may be suitable, including but not limited to, silicone polymers, hydrocarbons, linear fatty alcohols, esters of fatty acids or glyceride derivatives, mixtures thereof, or complexes thereof, and the like.

Examples of waxes for soft solid/stick applications include, but are not limited to, castor wax, beeswax, carnauba and candelilla waxes, which are of vegetable origin and mineral waxes from fossil remains other than petroleum. Montan wax, which is an example of mineral wax, includes non-glyceride esters of carboxylic acids, hydrocarbons and other constituents. Other naturally available waxes include spermeceti wax, ozokerite, ceresin, baysberry, synthetic waxes, paraffin wax, silicone waxes or mixtures thereof, and the like.

The term “oils” herein, refers to an organic compound which at about 20° C. is both liquid and water-insoluble. In the context of the invention, insolubility in water is understood to be a solubility of less than 10 w/w % at about 20° C. Typically, the solubility of less than 1 w/w %, more particularly less than 0.1 w/w %, in particularly less than 0.01 w/w %.

Examples of oil emollients include, but are not limited to, ester oils, an ether oil or a mixture thereof, and the like. The term “ester oil” herein, refers to oils as above-defined, which comprises at least one ester group. This means that also esters of carbonic acids are ester oils according to the invention. Further, the term “ether oil” herein, refers to oils as above-defined, which comprises at least one ether group.

Examples of oils include, but are not limited to, glycerides (such as e.g., triglycerides), hydrocarbons (such as e.g., petrolatum); silicone oils (such as e.g., dimethicone), dialkyl ethers, alkyl esters, dialkyl carbonates, dialkyl tartrates, natural oils (such as vegetable oils), and the like. In dialkyl ethers, alkyl esters and dialkyl carbonates such as dicaprylyl carbonate, ethylhexyl carbonate, dihexyl carbonate; the alkyl groups may be straight or branched and independently from each other typically have C₂-C₁₆ atoms. Generally, at least one of said alkyl groups has at least 6 carbon atoms, typically at least 8 carbon atoms. Optionally the oil may be an oil that has further functions such as an oil soluble sunscreen. Mixtures of oil components may also be used.

Typically, the emollient includes, but is not limited to, a dialkyl ether such as dicaprylyl ether, or an alkyl ester, C₁₂-C₁₆ alkyl benzoates or C₁₀-C₁₆ dialkyl tartrates. Generally, the emollient is a C₁₀-C₁₆ dialkyl tartrate, specifically C₁₂-C₁₃ dialkyl tartrate; or a dicaprylyl ether, specifically dicaprylyl carbonate.

Suitably, emollients include, but are not limited to, C₁₂-C₁₃ dialkyl tartrate, dicaprylyl carbonate, triC₁₂-C₁₃ tartrate, marula oil, sunflower seed oil (Helianthus annuss), safflower seed oil (Carthamus tinctorius), coconut oil, capryl/caprylic triglyceride, linseed oil, canola oil, cottonseed oil, soybean oil, or mixtures thereof, and the like.

The preservative system contains personal care or cosmetic preservatives of one or more preservative including, but is not limited to, benzoic acid or salt thereof, benzyl alcohol, sorbic acid or salt thereof, parabens, dehydroacetic acid, sodium dehydroacetate, bronopol, triclosan, imidazolidinyl urea, PHMB (polyhexamethylene biguanide), phenoxyethanol, DMDMH (1,3-Dimethylol-5,5-dimethylhydantoin), isothiazolones, chlorhexidine, diazolidinyl urea, chlorphenesin, sodium hydroxymethylglycinate, benzethonoium chloride, ethylhexylglycerol, IPBC (iodopropynyl butylcarbamate), salicylic acid or salt thereof, sodium benzoate, calcium gluconate, D-glucono1,5-lactone, glycerin, or natural acid or any preservative from the Annex V preservative list, and the like.

Typically the preservative system is present in an amount between 0.01 w/w %-10 w/w %; whereby the total amount of all preservatives used is within the provided range; based on the total weight of the composition. Suitably, the preservative system is present in an amount between 0.5 w/w %-5 w/w %, based on the total weight of the composition.

Suitably, the preservative system includes, but not limited to, one or more of a dehydroacetic acid, benzyl alcohol, sorbic acid, salicyclic acid, benzoic acid, sodium benzoate, calcium gluconate, D-glucono1,5-lactone, glycerin, phenoxyethanol, DMDMH, sodium dehydroacetate, iodopropynyl butylcarbamate (IPBC)and the like.

Typically the aqueous solvent is present in an amount wherein the concentrate composition totals up to 100 w/w %, based on the weight of the composition. Suitably, the aqueous solvent is present in an amount between 15 w/w %-55 w/w %.

Examples of aqueous solvents include, but not limited to, water, aqueous alcohols, ammonia water, acid solutions, salt solutions, water-miscible organic solvents, alkanolamines, glycol ethers, polyhydroxy alcohols (such as glycerin, diglycerin, propylene glycol, dipropylene glycol, sorbitol, pantenol and sugar); urea, alpha-hydroxy acid and its salt; and low molecular weight polyethylene glycols with molecular weight less than 20,000; water soluble or dispersible polymers; alkanolamines, or glycol ethers or mixtures thereof; and the like. Generally, the solvent used is water or an aqueous alcohol.

The term “aqueous alcohol” herein, refers to saturated, unsaturated, straight or branched hydrocarbon chain having C₂-C₄ carbon atoms and at least one hydroxyl groups, wherein the hydrocarbon chain may optionally comprise one or several heteroatoms (such as oxygen or sulfur).

Although not necessary, humectants may be added, if desired, without disturbing the properties within the cleansing concentrate composition. Suitable humectants useable as component can essentially be any known humectant that will not cloud or discolor the concentrate.

Examples of humectants include, but are not limited to, amino acids, pyrrolidone carboxylic acid, lactic acid and salts thereof, lactitol, urea and urea derivatives, uric acid, glucosamine, creatinine, cleavage products of collagen, chitosan or chitosan salts/derivatives and, in particular, polyols and polyol derivatives, for example glycerol, diglycerol, triglycerol, polyglyerin, such as polyglycerin-6, ethylene glycol, propylene glycol, butylene glycol, erythritol, 1,2,6-hexanetriol, polyethylene glycols, sugars and sugar derivatives (e.g. fructose, glucose, maltose, maltitol, mannitol, inositol, sorbitol, sorbitol silanediol, sucrose, trehalose, xylose, xylitol, glucuronic acid and salts thereof), ethoxylated sorbitol (Sorbeth-6, Sorbeth-20, Sorbeth-30, Sorbeth-40), honey and hydrogenated honey, hydrogenated starch hydrolyzates and mixtures of hydrogenated wheat protein and PEG-20-acetate copolymer, and the like.

Accordingly, if a humectant is desired, the humectant may be a polyol, in particular propylene glycol, glycerol, diglycerol and/or triglycerol. Generally, the humectant may be present in an amount between 10 w/w %-45 w/w %, based on the weight of the composition; or the humectant may be present in an amount <10 w/w %, based on the weight of the composition. Furthermore, the cleaning concentrate composition may be essentially free of a humectant.

The cleansing concentrate composition of the present invention may contain:
(a) the first emulsifier comprises a polyglyceryl fatty ester and wherein the polyglyceryl fatty esters comprises one or more of a polyglyceryl-10 decaoleate, polyglyceryl-3 monostearate, polyglyceryl-6 distearate, polyglyceryl-3 tetraoleate;
(b) the second emulsifier comprises a polyglyceryl fatty ester, wherein the polyglyceryl fatty esters comprises polyglyceryl-10 stearate, polyglyceryl-10 oleate, polyglyceryl-10 dipalmitate, polyglyceryl-10 caprylate/caprate, or polyglyceryl-10 laurate;
(c) the emollient comprises an oil, wherein the oil comprises an ester oil, an ether oil or mixtures thereof;
(d) the preservative system comprising one or more of a dehydroacetic acid, benzyl alcohol, sorbic acid, salicylic acid, benzoic acid, sodium benzoate, calcium gluconate, D-glucono1,5-lactone, glycerin, phenoxyethanol, DMDMH (1,3-Dimethylol-5, 5-dimethylhydantoin), sodium dehydroacetate, iodopropynyl butylcarbamate (IPBC);
(e) the aqueous solvent comprises water; and
   wherein the concentrate composition totals up to 100 w/w %, based on the weight of the composition.

If desired, the cleansing concentrate composition may comprise:
(a) the first emulsifier comprises a polyglyceryl fatty ester and wherein the polyglyceryl fatty esters comprises one or more of a polyglyceryl-10 decaoleate, polyglyceryl-3 monostearate, polyglyceryl-6 distearate, or polyglyceryl-3 tetraoleate;
(b) the second emulsifier comprises a polyglyceryl fatty ester, wherein the polyglyceryl fatty esters comprises polyglyceryl-10 stearate, polyglyceryl-10 oleate, polyglyceryl-10 d ipalm Rate, polyglyceryl-10 caprylate/cap rate, or polyglyceryl-10 lau rate;
(c) an emollient comprising a C₁₀-C₁₆ dialkyl tartrate;
(d) a preservative system comprising one or more of a dehydroacetic acid, benzyl alcohol, sorbic acid, salicyclic acid, benzoic acid, sodium benzoate, calcium gluconate, D-glucono1,5-lactone, glycerin, phenoxyethanol, DMDMH (1,3-Dimethylol-5,5-dimethylhydantoin), sodium dehydroacetate, iodopropynyl butylcarbamate (IPBC);
(e) an aqueous solvent comprising of water; and
   wherein the concentrate composition totals up to 100 w/w %, based on the weight of the composition.

Optionally, the cleansing concentrate for use as a concentrate base, lotion, solution, cream, gel, solid, or wet wipe saturant, further comprises at least one additional ingredient or components such as cosmetic active, pharmaceutical ingredient, personal care ingredient including, but not limited to, co-surfactant, fragrance, viscosity modifier, moisturizing agent, antioxidant, pharmaceutical ingredient, booster, potentiator, biocidal agent, cosmetic active, sun-protectant, UV-protectant, biological active, occlusive agent ,or combinations thereof, and the like. Any additional component may be added the composition without disturbing the properties and end-user formulation for which the cleansing concentrate composition may be used.

Stability testing of the cleansing concentrate composition was performed at about 25° C. to about 50° C., respectively for 1 month; with results showing complete stability of the composition after the 1 month period. Specifically, the stability testing was performed at 25° C., 37.5° C., and 50° C. respectively, for more than 2 wks.

In another aspect, the present invention provides a cleansing concentrate composition as described or upon dilution with an aqueous solvent such as water or an aqueous alcohol; may be used to formulate suitable end-user personal care, cosmetic and hygiene products. For example, the present invention provides a wet wipe impregnating formulation for personal care, cosmetics or hygiene products comprising (a) a cleansing concentrated composition according to the present invention, and (b) an aqueous solvent comprising water, an aqueous alcohol or a mixtures thereof.

Typically, the cleansing concentrate composition may be diluted as desired for use in an end-use formulation for end-user personal care, cosmetic and hygiene products such as a wet wipe impregnating formulation, to contain 0.1 w/w %-50 w/w % of the cleansing concentrate. Desirably, the cleansing concentrate composition can be further diluted to contain 0.5 w/w %-30 w/w % of the concentrate in suitable end-user personal care, cosmetic and hygiene products, such as wet wipes, base for makeup remover, rinse off cleansing solution, cream or serum composition, make-up removal wipe or lotion, skincare lotion, cream, shampoo, moisturizer, cleanser, and the like.

Examples of end-user personal care, cosmetic and hygiene products include, but not limited to, a personal care, cosmetic or hygiene wet wipe (i.e. baby wipes, cosmetic wipes, hygiene wipes, consumer care wipes), and other wet wipe containing said formulations (such as antimicrobial or disinfectant wet wipes); and cosmetics, personal and other skincare products (such as antimicrobial or hygiene solutions, a micellar water solution, gels, solids, a moisturizing cream and/or lotion, skin care creams, lotions and/or milks, a cleanser solution (facial or body), a shampoo solution, a conditioning solution, a make-up removal or cosmetic solution or lotion, scalp/hair care products, and the like).

Suitably, the cleansing concentrate composition may be used as is or upon dilution to formulate end-user personal care, cosmetic and hygiene products. For example wet wipes, hygiene wipes, baby wipes, solutions, and facial cleansers. Hygiene wipes also includes, but not limited to, wipes for domestic and industrial use, ready to use (RTU) sprays for domestic and industrial use and detergency and antimicrobial systems, making use of the formulations low toxicity and irritancy.

The desired formulation may be a wet wipe impregnating formulation for personal care, cosmetics or hygiene products comprising (a) a cleaning concentrated composition according to the present invention, and (b) water, an aqueous alcohol or a mixture thereof.

For example, the concentrate composition may be used to form a wet wipe saturating formulation, used to saturate or impregnate a wet wipe substrate. The wet wipe saturating formulation can be the cleansing concentrate or a dilute form of the cleansing concentrate, diluted with an aqueous solvent. Generally, the solvent mixed with concentrate may be water or an aqueous alcohol. The mixture of the water with the oil-in-water concentrate will form an oil-in-water emulsion upon dilution.

This wet wipe saturating formulation will typically contain 0.1 w/w %-50 w/w % by weight of the cleansing concentrate composition. Suitably, the wet wipe saturating formulation will contain 0.5 w/w %-30 w/w % by weight of the cleansing concentrate composition.

The wet wipe substrate used for the wet wipe impregnating formulation includes but is not limited to, a woven, nonwoven material, mixtures thereof, and the like; used for personal care, cosmetic, hygiene and consumer care end user products, or others suitable for use herein.

Examples of wet wipe substrates include, but are not limited to, woven, nonwoven materials, or natural and synthetic sponges. Essentially any nonwoven material may be used. Exemplary nonwoven materials may include, but are not limited to meltblown, coform, spunbond, airlaid, hydroentangled nonwovens, spunlace, bonded carded webs, and laminates thereof.

Optionally, the nonwoven material may be laminated with a polymer films or an absorbent or non-absorbent fibers. The fibers used to prepare a nonwoven wipe substrate include, but are not limited to, cellulosic fibers, thermoplastic fibers, polyester, polyamide (e.g. nylon), acrylic and polypropylene fibers and mixtures thereof. The fibers may also be continuous fibers, discontinuous fibers, staple fibers and mixtures thereof.

The wet wipe impregnating formulation is impregnated into the wipe substrate such that the wipe is pre-moistened and may express or release the formulation on to a surface as the wipe is run across the surface. Generally, the formulation is saturated into the wipe such that the wipe may release the formulation to the substrate of a surface through a wiping action.

The amount of the wet wipe impregnating formulation used to impregnate a wipe substrate may vary over a wide range, depending on the particular wipe substrate and the wipe's intended use. The desired wipe may further comprise a wet wipe impregnating formulation and the wipe substrate in a weight ratio of about 20:1 to 0.5:1, suitably 10:1 to 1:1.

The following examples illustrate the invention without limitation. All parts and percentages are given by weight unless otherwise indicated.

It will be understood that each of the elements described in the examples below, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims.

The foregoing embodiments are presented by way of example only; the scope of the present disclosure is to be limited only by the following claims.

### EXAMPLE 1

TABLE 1
A cold-processible wet wipe concentrate, base for makeup remover,
rinse off cleansing solution, cream or serum composition.

| | w/w % conc. |
|---|---|
| Ingredients phase A | |
| Polyglyceryl 10-caprate/caprylate in 25% water | 12% |
| polyglyceryl-10 decaoleate | 7% |
| DiC₁₂-C₁₃ alkyl tartrate | 55% |
| Ingredients phase B | |
| water | 25.3% |
| Benzyl alcohol, salicylic acid, sorbic acid, glycerin | 0.7% |

The polyglyceryl esters in phase A were first mixed together, then the emollient was slowly added while homogenizing phase A, after which phase B was slowly added to phase A and the solution was homogenized. In this formulation, the polyglyceryl ester(s) can be substituted for polyglyceryl-10 laurate, polyglyceryl-10 oleate, or polyglyceryl-3 tetraoleate. The emollient can be substituted for any other emollient as previously described.

### EXAMPLE 2

TABLE 2
A concentrate base, make-up solution, cleanser,
wet wipe or lotion composition

| | w/w % conc. |
|---|---|
| Ingredients phase A | |
| Polyglyceryl 10-caprate/caprylate in 25% water | 12% |
| polyglyceryl-10 decaoleate | 7% |
| DiC₁₂-C₁₃ alkyl tartrate | 55% |
| Benzyl alcohol, dehydroacetic acid | 1% |
| Ingredients phase B | |
| Water | 25% |

The polyglyceryl esters in phase A were first mixed together, then the dialkyl tartrate was slowly added, while homogenizing phase A; after which phase B was slowly added to phase A, then mixture was homogenized. Citric acid was used to adjust the pH of the solution having a pH of 4. If diluted ×10 a make-up lotion/wipe formulation may be formed, and if diluted ×25 a baby wipe/lotion formulation may be formed.

### EXAMPLE 3

TABLE 3
A concentrate base for make-up removal
wipe or lotion composition

| | w/w % conc. |
|---|---|
| Ingredients phase A | |
| Polyglyceryl 10-caprate/caprylate in 25% water | 12% |
| polyglyceryl-10 decaoleate | 7% |
| Marula oil | 65% |
| Ingredients phase B | |
| Water | 15.2% |
| Benzyl alcohol, salicylic acid, sorbic acid | 0.8% |

The polyglyceryl esters in phase A were first mixed together, then dialkyl tartrate was slowly added, while homogenizing Phase A; after which phase B were slowly added to phase A, and the mixture was homogenized.

### EXAMPLE 4

TABLE 4
A concentrate base, skin care, moisturizer,
lotion or shampoo solution

| | w/w % conc. |
|---|---|
| Ingredients phase A | |
| Polyglyceryl 10-caprate/caprylate in 25% water | 12% |
| polyglyceryl-10 decaoleate | 7% |
| Coconut oil | 55% |
| Ingredients phase B | |
| Water | 25.2% |
| Benzyl alcohol, salicylic acid, sorbic acid | 0.8% |

The polyglyceryl esters in phase A were first mixed together, then dialkyl tartrate was slowly added, while homogenizing Phase A; after which phase B were slowly added to phase A, and the mixture was homogenized. In concentrated form, the example above can be used for solid skin care products and when diluted the composition can be used for moisturizing, creams, lotions, or shampoo solutions.

### EXAMPLE 5

TABLE 5
A concentrate base, cleanser or moisturizer or lotion composition

| | w/w % conc. |
|---|---|
| Ingredients phase A | |
| Polyglyceryl 10-oleate in 25% water | 12% |
| polyglyceryl-10 decaoleate | 7% |
| Dicaprylyl Carbonate | 55% |
| Ingredients phase B | |
| Water | 25% |
| Sodium Benzoate | 1% |

The polyglyceryl esters in phase A were first mixed together, then dialkyl ether was slowly added, while homogenizing Phase A; after which phase B were slowly added to phase A, and the mixture was homogenized.

### EXAMPLE 6

TABLE 6
A cold-processible wet wipe concentrate, make-up remover base,
cream, serum, or rinse-off cleansing solution composition.

| | 6a w/w % | 6b w/w % |
|---|---|---|
| Ingredients phase A | | |
| Polyglyceryl-10 decaoleate | 7% | 7% |
| Blend lactobacillus, milk | | 1% |
| solid, soybean oil | | |
| ferment, cyclodextrin | | |
| Blend calyx extract, | 3% | — |
| caprylic/capric triglyceride, | | |
| β-carotene, α-tocopherol | | |
| Linseed oil, canola oil, morus | — | 1% |
| nigra leaf extract, pearl | | |
| xtract, α-tocopherol | | |
| Ingredients phase B | | |
| Polyglyceryl-10 laurate | 3% | 3% |
| in 25% water | | |
| Cotton seed oil | 50% | 50% |
| Ingredients phase C | | |
| Water | Qs to 100% | Qs to 100% |
| Benzoic acid | 0.9% | 0.9% |
| niacinamide | — | 1% |
| Ingredients phase D | | |
| Citric acid monoglyceride | Qs pH 4.0 | Qs pH 4.0 |
| monohydrate | | |

The blend active in phase A was first dissolved in the emulsifier. The polyglyceryl ester in phase B were first mixed together with phase A, then the emollient was slowly added while homogenizing phase B, after which phase C was slowly added to the solution and the solution was homogenized. Active in phase D was used to adjust the pH of the solution having a pH of 4.

### EXAMPLE 7

TABLE 7
A solid skin care, wax, cream or lotion concentrate composition.

| | w/w % |
|---|---|
| Ingredient phase A | |
| Polyglyceryl-10 caprylate/caprate | 3% |
| Polyglyceryl-10 decaoleate | 7% |
| Coconut oil | 55% |
| Ingredient phase B | |
| Water | Qs to 100% |
| Benzyl alcohol, salicylic acid, sorbic acid | 0.8% |

Composition shown in Table 7 can be diluted for use as a wipe impregnating formulation with the use of heat. The polyglyceryl esters in phase A were first mixed together, then the emollient was heated to about 60° C. and slowly added, while homogenizing Phase A; after which phase B were slowly added to phase A, and the mixture was homogenized. In concentrated form, the example above can be used for solid skin care products and when diluted the composition can be used for moisturizing, creams, lotions, or shampoo solutions.

### EXAMPLE 8

TABLE 8
A make-up removal wipe or lotion composition.

| | 8a w/w % | 8b w/w % |
|---|---|---|
| Ingredients phase A | | |
| Polyglyceryl-10 | 12% | 12% |
| caprate/caprylate | | |
| (25% in water) | | |
| Polyglyceryl-10 decaoleate | 7% | 7% |
| Polyglyceryl-10 laurate | 2% | — |
| DiC₁₂-C₁₃ tartrate | 52% | — |
| Marula oil | — | 65% |
| Ingredient phase B | | |
| Water | Qs to 100% | Qs to 100% |
| Benzyl alcohol, | 0.8% | — |
| salicylic acid, sorbic acid | | |
| Sodium benzoate | — | 0.3% |
| Phenoxyethanol | — | 0.7% |
| Ingredient phase C | | |
| Citric acid monohydrate | — | Qs to pH 4 |

The polyglyceryl esters in phase A were first mixed together, then dialkyl tartrate was slowly added, while homogenizing Phase A; after which phase B were slowly added to phase A, and the mixture was homogenized.

### EXAMPLE 9

TABLE 9
A cold-processible make-up solution, cleanser,
wet wipe or lotion base composition.

| | 9a w/w % | 9b w/w % |
|---|---|---|
| Ingredient phase A | | |
| Polyglyceryl-10 | 12% | — |
| caprate/caprylate in | | |
| 25% water | | |
| Polyglyceryl-10 decaoleate | 7% | 7% |
| Polyglyceryl-10 laurate | — | 12% |
| in 25% water | | |
| Cottonseed oil | | 60% |
| DiC₁₂-C₁₃ tartrate | 55% | — |
| Benzoic acid | — | 0.5% |
| Benzyl alcohol, | 1% | — |
| dehydroacetic acid | | |
| Ingredient phase B | | |
| Water | Qs to 100% | Qs to 100% |
| Ingredient phase C | | |
| Citric acid monohydrate | Qs to pH 4 | Qs to pH 4 |

The polyglyceryl esters in phase A were first mixed together, then the dialkyl tartrate was slowly added, while homogenizing phase A; after which phase B was slowly added to phase A, then mixture was homogenized. Citric acid was used to adjust the pH of the solution having a pH of 4. If diluted ×10 in an aqueous solution a make-up lotion/wipe formulation may be formed, and if diluted ×25 a baby wipe/lotion formulation may be formed.

### EXAMPLE 10

TABLE 10
Wet wipe impregnating formulation for
use in personal care wipe applications.

| | 10a w/w % | 10b w/w % | 10c w/w % |
|---|---|---|---|
| Ingredient phase A | | | |
| Any formulation from Table | 0.5-30% | 0.5-30% | 0.5-30% |
| 1-9 | | | |
| water | Qs to 100% | Qs to 100% | Qs to 100% |
| galactoarabinan | — | — | 2% |
| D-Glucono-1,5-Lactone, | 1% | 1% | 1% |
| Sodium benzoate, and | | | |
| Calcium gluconate | | | |
| triethanolamine | Qs to pH 5.5 | Qs to pH 5.5 | Qs to pH 5.5 |

The ingredients in Phase A were mixed together at room temperature until the preservative was fully solubilized. Wet wipe impregnating formulation for use in personal care wipe applications. Formulation listed in table 1 can be diluted to 0.5 w/w % to 30.0 w/w % in an aqueous solvent (i.e. water) and a preservative. The ingredients can be mixed together at room temperature until the preservative was fully solubilized. A pH regulator can be used by the formulator to adjust to desired pH.

### EXAMPLE 11

TABLE 11
Dilution of concentrate for serum, cream or lotion applications

| | 11a w/w % | 11b w/w % |
|---|---|---|
| Ingredient phase A | | |
| polyglyceryl-10 laurate | 2-5% | — |
| Polyglyceryl-10 dipalmitate | — | 3% |
| water | Qs to 100% | Qs to 100% |
| Ingredients phase B | | |
| All formulations listed | 0.5-30% | 0.5-30% |
| in any one of Table 1-9 | | |
| D-Glucono-1,5-Lactone, | 1% | — |
| Sodium benzoate, and | | |
| Calcium gluconate | | |
| (Geogard Ultra ™)¹ | | |
| Benzyl alcohol | — | 1% |
| triethanolamine | Qs to pH 5.5 | Qs to pH 5.5 |

The ingredients in Phase A were mixed together at room temperature until dissolution of the emulsifier. Phase A was slowly added to phase B under agitation until full dissolution of product. Dilution of concentrate for use in lotion applications. Formulation listed in table 1 can be diluted to 0.5 w/w % to 30.0 w/w % in an aqueous solvent (i.e. water), an surfactant and a preservative. The ingredients can be mixed together at room temperature until the preservative was fully solubilized. A pH regulator can be used by the formulator to adjust to desired pH. Can substitute emollient/oil for alternative. Namely, dicaprylyl carbonate, tri C12-13 alkyl tartrate, marula oil, Helianthus annuus (sunflower) seed oil, Carthamus tinctorius (safflower) seed oil, coconut oil, capryl/caprilic triglyceride.

Dilution of concentrate for use in cream or serum applications. Dilution of concentrate for use in lotion applications. Formulation listed in table 1 can be diluted to 0.5 to 30.0% in an aqueous solvent (i.e. water), an emulsifier/stabilizer and a preservative. The ingredients can be mixed together at room temperature until the preservative was fully solubilized. A pH regulator can be used by the formulator to adjust to desired pH.

While the invention has been described above with references to specific embodiments thereof, it is apparent that many changes, modifications and variations can be made without departing from the invention concept disclosed herein. Accordingly, it is intended to embrace all such changes, modifications, and variations that fall within the spirit and broad scope of the appended claims.

What is claimed is:

1. A cleansing concentrate composition for use as a lotion, solution, cream, gel, solid or wet wipe saturant comprising;
(a) a first emulsifier selected from group consisting of a polyglyceryl fatty ester, an ethoxylated fatty alcohol, an ethoxylated fatty acid, an ethoxylated sorbitan ester and a mixture thereof, having an HLB between 1 and 9;
(b) a second emulsifier selected from group consisting of a polyglyceryl fatty ester, an ethoxylated fatty alcohol, an ethoxylated fatty acid, an ethoxylated sorbitan ester and a mixture thereof having an HLB between 9-16;
(c) an emollient;
(d) a preservative system; and
(e) an aqueous solvent.

2. The cleansing concentrate composition according to claim 1, wherein the first and second emulsifier are each present in an amount between 2 w/w %-30 wiw %, based on the weight of the composition; the emollient is present in an amount between 15 w/w %-80 w/w %; based on the weight of the composition;
the preservative system present in an amount from 0.01 w/w %-10 w/w %, based on the weight of the composition; and the aqueous solvent present in an amount from 12 w/w %-55 w/w %, based on the weight of the composition.

3. The cleansing concentrate composition according to claim 2, wherein the first and second emulsifier are each present in an amount between 3 w/w %-20 w/w %, based on the weight of the composition; the emollient is present in an amount between 40 w/w %-80 wiw %; based on the weight of the composition;
and the preservative system present in an amount from 0.5 w/w %-5 w/w %, based on the weight of the composition.

4. The cleansing concentrate composition according to claim 1, wherein the first or second emulsifier is selected from group consisting of a polyglyceryl fatty ester, an ethoxylated fatty alcohol, an ethoxylated fatty acid and mixtures thereof.

5. The cleansing concentrate composition according to claim 4, wherein the ratio of the first emulsifier to the second emulsifier is about 1:10 to 10:1.

6. The cleansing concentrate composition according to claim 4, wherein the first and second emulsifier each comprises one or more compound of a polyglyceryl fatty ester.

7. The cleansing concentrate composition according to claim 1, wherein the polyglyceryl fatty ester is derived from (1) a polyglycerol component built up from 2 to 12 molecules of glycerol, based on an average, and (2) a fatty acid comprising a caprylic acid, capric acid, lauric acid, myristic acid, palmitic acid, stearic acid, arachidic acid, behenic acid, lignoceric acid, cerotic acid, oleic acid, decaoleic acid or mixtures thereof.

8. The cleansing concentrate composition according to claim 7, wherein the (a) first emulsifier comprises a polyglyceryl fatty ester wherein the polyglyceryl fatty esters comprises one or more of a polyglyceryl-10 decaoleate, polyglyceryl-3 monostearate, polyglyceryl-6 distearate, or polyglyceryl-3 tetraoleate, and the (b) second emulsifier comprises a polyglyceryl fatty ester, wherein the polyglyceryl fatty esters comprises polyglyceryl-10 stearate, polyglyceryl-10 oleate, polyglyceryl-10 dipalmitate, polyglyceryl-10 caprylate/caprate, or polyglyceryl-10 laurate.

9. The cleansing concentrate composition according claim 1, wherein the emollient comprises a wax or an oil.

10. The cleansing concentrate composition according to claim 9, wherein the oil comprises an ester oil, an ether oil, or a mixture thereof.

11. The cleansing concentrate composition according to claim 10, wherein the ester oil comprises a dialkyl tartrate, dialkyl carbonate, marula oil, sunflower seed oil, safflower seed oil, coconut oil, capryl/caprylic triglyceride, linseed oil, canola oil, cottonseed oil, soybean oil, or mixture thereof.

12. The cleansing concentrate composition according claim 1, wherein the preservative system comprises one or more preservative of a benzoic acid or salt thereof, benzyl alcohol, sorbic acid or salt thereof, parabens, dehydroacetic acid, bronopol, triclosan, imidazolidinyl urea, PHMB (polyhexamethylene biguanide), phenoxyethanol, DMDMH (1,3-Dimethylol-5,5-dimethylhydantoin), isothiazolones, chlorhexidine, diazolidinyl urea, chlorphenesin, sodium hydroxymethylglycinate, benzethonoium chloride, ethylhexylglycerol, 1PBC (iodopropynyl butylcarbamate), salicylic acid or salt thereof, sodium benzoate, calcium gluconate, D-glucono1,5-lactone, glycerin, or natural acid.

13. The cleansing concentrate composition according claim 1, wherein the aqueous solvent comprises water, aqueous alcohols, ammonia water, acid solutions, salt solutions, water-miscible organic solvents, or mixtures thereof.

14. The cleansing concentrate composition according to claim 13, wherein the aqueous solvent comprises water.

15. The cleansing concentrate composition according to claim 1, further comprising a humectant, wherein the humectant is present in an amount from 10 w/w %-45 w/w %, based on the total weight of the composition.

16. The cleansing concentrate composition according to claim 1, further comprising a humectant, wherein the humectant is present in an amount less than 10 w/w %.

17. The cleansing concentrate composition according to claim 1, wherein the composition is essentially free of a humectant.

18. The cleansing concentrate composition according to claim 1, wherein:
a) the first emulsifier comprises a polyglyceryl fatty ester and wherein the polyglyceryl fatty esters comprises one or more of a polyglyceryl-10 decaoleate, polyglyceryl-3 monostearate, polyglyceryl-6 distearate, polyglyceryl-3 tetraoleate;
(b) the second emulsifier comprises a polyglyceryl fatty ester, wherein the polyglyceryl fatty esters comprises polyglyceryl-10 stearate, polyglyceryl-10 oleate, polyglyceryl-10 dipalmitate, polyglyceryl-10 caprylate/caprate, or polyglyceryl-10 laurate;
(c) the emollient comprises an oil, wherein the oil comprises an ester oil, an ether oil or mixtures thereof;
(d) the preservative system comprising one or more of a dehydroacetic acid, benzyl alcohol, sorbic acid, salicylic acid, benzoic acid, sodium benzoate, calcium gluconate, D-gluconol,5-lactone, glycerin, phenoxyethanol , DMDMH (1,3-Dimethylol-5,5-dimethylhydantoin), sodium dehydroacetate, or iodopropynyl butylcarbamate (IPBC);
(e) the aqueous solvent comprises water; and
wherein the concentrate composition totals up to 100 w/w %, based on the weight of the composition.

19. The cleansing concentrate composition according to claim 1, further comprising a co-surfactant, fragrance, viscosity modifier, moisturizing agent, antioxidant, active pharmaceutical ingredient, booster, potentiator, biocidal agent, cosmetic active, sun-protectant, UV-protectant, biological active, occlusive agent or mixtures thereof.

20. A wet wipe impregnating formulation for personal care, cosmetics or hygiene products comprising (a) a concentrated composition according to claim 1, and (b) an aqueous solvent comprising water, an aqueous alcohol or a mixtures thereof.

21. The wet wipe impregnating formulation according to claim 20, wherein the formulation comprises 0.1 w/w %-50 w/w %, by weight of the concentrate composition, based on the weight of the formulation.

22. The wet wipe impregnating formulation according to claim 21, wherein the formulation comprises between 0.5 w/w %-30 w/w %, by weight of the concentrate composition, based on the weight of the formulation.

23. The wet wipe according to claim 20, wherein the wipe substrate comprises a woven, a nonwoven material or mixtures thereof.

24. The wet wipe according to claim 23, wherein the wipe comprises the wet wipe impregnating formulation and the wipe substrate in a weight ratio of about 10:1 to 1:1.
